(19) United States
(12) Patent Application Publication
(10) Pub. No.: US 2010/0311072 A1
(43) Pub. Date: Dec. 9, 2010

(54) FLUORESCENCE POLARIZATION BINDING ASSAY FOR CHARACTERIZING GLUCOKINASE LIGANDS

(75) Inventors: Ramakrishna Seethala, West Windsor, NJ (US); Jessica Leihwa Dong, Brisbane, CA (US); Kevin M. O'Malley, Chalfont, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/864,745

(22) PCT Filed: Jan. 28, 2009
(86) PCT No.: PCT/US09/32210

§ 371 Date: Jul. 27, 2010

Related U.S. Application Data
(60) Provisional application No. 61/023,945, filed on Jan. 28, 2008.

(51) Int. Cl. G01N 33/53; C12Q 1/68

(52) U.S. Cl. 435/6; 435/7.8; 435/7.6

(57) ABSTRACT

The subject matter disclosed and claimed herein concerns measuring the binding affinity of glucokinase (“GK”) using a fluorescence polarization (“FP”) assay. The FP method includes use of modified GK ligands bound to a fluorescent label. Binding affinity is determined by measuring displacement of fluorescent ligand by the known or suspected GK ligands. The subject matter disclosed and claimed herein provides a robust high-throughput FP assay for the determination of binding affinity of ligands to glucokinase. The FP binding assay displayed both glucose and nucleotide dependence, and a useful dynamic range. The binding IC₅₀ data correlated well with GK activation EC₅₀ data.

This application claims priority to U.S. Provisional Application No. 61/023,945, filed on Jan. 28, 2008. The subject matter described and claimed therein is incorporated by reference herein.

### FIELD

The subject matter disclosed and claimed herein relates to assays and methods for evaluating the binding characteristics of glucokinase (“GK”) ligands. Specifically, the subject matter disclosed and claimed herein concerns measuring the binding affinity of GK using a fluorescence polarization (“FP”) assay. The FP method includes use of modified GK ligands bound to a fluorescent label (e.g., fluorescein). Binding affinity is determined by measuring displacement of fluorescent ligand by the known or suspected GK ligands. The subject matter disclosed and claimed herein provides a robust high-throughput FP assay for the determination of binding affinity of ligands to glucokinase. The FP binding assay displayed both glucose and nucleotide dependence, and a useful dynamic range. The binding IC₅₀ data correlated well with GK activation EC₅₀ data.

### BACKGROUND

All references, including patents and patent applications, are hereby incorporated by reference in their entireties.

### Glucokinase

Glucokinase (“GK”) is a hexokinase family member and catalyzes the first step in glycolysis. GK is one of the four mammalian glucose phosphorylating isoenzymes and serves as a glucose sensor in specific tissues requiring “glucose sensing”, such as the liver, pancreatic β-cells, hypothalamus, pituitary, and K- and L-enteroendocrine cells of the GI tract (Matschinsky, F. M., “Glucokinase as glucose sensor and metabolic signal generator in pancreatic β cells and hepatocytes”, Diabetes, 39:647-652 (1990)). Unlike other hexokinase family members, GK has a distinctive structure, enzymatic activity and tissue localization. GK is the only family member known to have an allosteric activation site. Activation of GK by overexpression, genetic mutations, and small molecule allosteric activators have all been shown to increase insulin secretion and decrease whole body glucose load. This activity suggests agents that bind to this allosteric site, and activate GK, could serve as specific anti-diabetic agents.

GK's significant role in the control of blood glucose levels is underscored by research using transgenic animals and in humans possessing GK mutations. For example, pancreatic or liver-specific GK knockout mice display hyperglycemia (Postic, C. et al., “Dual roles for glucokinase in glucose homeostasis as determined by liver and pancreatic β cell specific gene knockouts using Cre recombinase.” J. Biol. Chem., 274:305-315 (1999)). Overexpression of GK leads to lower fasting blood glucose levels and resistance to the development of high fat diet-induced diabetes (Niswender, K. D. et al., “Cell-specific expression and regulation of a glucokinase gene locus transgene”, J. Biol. Chem., 272:22564-22569 (1997); Shiota, M. et al., “Glucokinase gene locus transgenic mice are resistant to the development of obesity-induced type 2 diabetes”, Diabetes, 50:622-629 (2001)). In humans, naturally occurring inactivating and activating mutations in the gene encoding GK were reported to cause maturity onset diabetes of the young type-2 (MODY2) (Vionnet, N. et al., “Nonsense mutation in the glucokinase gene causes early onset non-insulin-dependent diabetes mellitus”, Nature, 356:721-722 (1992); Froguel, P. et al., “Familial hyperglycemia due to mutations in glucokinase. Definition of subtype of diabetes mellitus”, N. Engl. J. Med., 328:697-702 (1993)), and persistent hyperinsulinemic hypoglycemia of infancy (PHHI) (Glasser, B. et al., “Familial hyperinsulinism caused by an activating glucokinase mutation”, N. Engl. J. Med., 338:226-230 (1998)).

The data reported in the literature reflect the importance of GK in regulating glucose homeostasis and suggest that pharmacological modulation (e.g., activation) of GK in patients having diabetes-related disorders could have therapeutic benefits. In recent years, several groups reported discovery of small molecules that enhance GK activity by apparent binding to the GK allosteric site. The identified compounds stimulate insulin secretion in a glucose-dependent manner in pancreatic β-cells and increase glucose uptake in rat hepatocytes. Additionally, it was observed that GK activators lowered blood glucose levels and improved glucose tolerance tests in wild type and Diet Induced Obese (DIO) mice (Grimsby, J. et al., “Allosteric Activators of Glucokinase: Potential Role in Diabetes Therapy”, Science, 301:370-373 (2003); Efanov, A. M. et al., “A Novel Glucokinase Activator Modulates Pancreatic Islet and Hepatocyte Function”, Endocrinology, 146:3696-3701 (2005); McKerrecher, D. et al., “Design of a potent, soluble glucokinase activator with excellent in vivo efficacy”, Bioorg. Med. Chem. Letters, 16:2705-2709 (2006)).

Despite the progress in identifying modulators of GK and in vitro assays, to fully characterize and evaluate GK modulators, there remains a need for a high-throughput binding assay.

### Fluorescence Polarization Techniques

In general, fluorescent polarization techniques are based on the principle that a fluorescent labeled compound when excited by linearly polarized light will emit fluorescence having a degree of polarization inversely related to its rate of rotation. Therefore, when a molecule, such as a tracer-ligand protein conjugate having a fluorescent label is excited with linearly polarized light, the emitted light remains highly polarized because the fluorophore is constrained from rotating between the time light is absorbed and emitted. When a “free” tracer compound (i.e., unbound to a ligand) is excited by linearly polarized light, its rotation is much faster than the corresponding tracer-ligand conjugate and the molecules are more randomly oriented, therefore, the emitted light is depolarized. Thus, fluorescent polarization provides a quantitative means for measuring the amount of tracer-ligand conjugate produced in a competitive binding assay.

As described above, when a fluorescent sample is excited with a polarized light, the emission from the sample is also polarized. In an FP assay, binding of a small fluorescent ligand like FL1 (low FP value) to a large protein molecule like GK results in an increase in FP signal. Fluorescence is read in parallel (S) and perpendicular (P) to the excitation plane in a multimode plate reader like ANALYST® (Molecular Devices, Sunnyvale, Calif.) and the FP is calculated according to the ratio:

$$mP = [ \frac{( S - P * G )}{( S + P * G )} ] \times 1000$$

where, mP is polarization given in milli polarization units, S and P are emissions parallel and perpendicular to the plane of polarization respectively and G is a correction factor called g-factor.

Application of FP techniques to modulators of GK provides an elegant and high volume assay for identifying agents that bind GK and further characterizing the binding properties of such agents.

### SUMMARY OF THE INVENTION

GK ligands (e.g., activators) bind at an allosteric site on GK which has the potential to reduce the Km for glucose and increase the reaction velocity. To determine the potencies of GK ligands, a fluorescence polarization (FP) binding assay was developed using a fluorescent ligand (“FL1”). FL1 was derived from a potent GK activator (“FL”), having an EC₅₀ of about 20 nM, which was covalently bound to a fluorescein group.

Generally, the FP binding assay described and claimed herein is a homogeneous assay wherein all of the reaction components are added into well(s) of a multiwell microtiter plate, incubated and, without further processing, the FP signal is measured using a device such as a microplate reader. The assay provides a rapid and efficient means for conducting GK binding assays.

There are several embodiments of the subject matter disclosed and claimed herein. For example, one embodiment is a method for measuring the binding characteristics of GK comprising: incubating assay buffer, glucokinase, a fluorescently labeled GK ligand, in a test reaction vessel; incubating assay buffer, glucokinase and a control media in a control reaction vessel; measuring the fluorescence in said reaction vessels; and comparing the fluorescence in the reaction vessels whereby a difference in fluorescence indicates binding to GK. Further embodiments include an assay buffer that comprises glucose, or AMP-PNP a non-hydrolyzable nucleotide analog of ATP; use of recombinant human GK (e.g., NCBI RefSeq NP_{—}277042, NP_{—}277043; NM_{—}033507, and NM_{—}033508) or any other GK may be used including human, rat, mouse, primate, canine, chicken, and GK or GK-like enzymes from lower organisms such as nematodes, yeast, and bacteria. Recombinant human GK is preferred. Mutants of human GK are also preferred. The GK sequences for a number of organisms are readily available from commercial and academic sources. Similarly, mutants of GK may be used as well to study those residues/structures involved with ligand binding and/or GK activation. Those having ordinary skill in the art would be able to construct a variety of GK mutants by employing traditional molecular biology techniques. Additional embodiments include use of multi-well reaction vessels and a variety of commercially available fluorescent labels such as fluorescein, BODIPY, rhodamine green, rhodamine red, tetramethylrhodamine, alexa fluor, OREGON GREEN®, and TEXAS RED®.

An additional embodiment includes a method for identifying or screening for agents that bind glucokinase (“GK”) comprising: a) incubating assay buffer, GK, a fluorescently labeled GK ligand, and a test compound dissolved in medium in a test reaction vessel; b) incubating assay buffer, GK, a fluorescently labeled GK ligand, and control media in a control reaction vessel; c) measuring the fluorescence in said reaction vessels; and d) comparing the fluorescence in the reaction vessels whereby a difference in fluorescence indicates binding to GK. This screening method may be described as a “competition” assay.

Such screening methods may farther include an assay buffer that comprises glucose and ATP analogs. The screening method may also include a positive control. That is, a known GK modulator may be included in the assay to serve as a positive benchmark while attempting to identify new GK modulators.

Similar to the first method described above, any species of GK or fragment thereof may be used in the screening methods. Recombinant human GK is preferred and mutant human UK sequences are contemplated herein. The methods described above were used to identify and/or characterize GK compounds having IC₅₀ values of from about 0.01 to about 2.5 μM in the presence of 12 mM glucose.

### DETAILED DESCRIPTION

### Description of the Figures

FIG. 1. Activation of GK activity by the fluorescent ligand FL1 and comparison with the parent compound FL. The GK activity was assayed by a coupled GK-G-6-P dehydrogenase tandem assay and monitoring by a thio NAD reduction in the presence of 2, 5, and 20 mM glucose. The EC₅₀ values are given in the parenthesis.

FIG. 2: Determination of binding affinity constants for GK at different glucose concentration in the absence (A) or presence (B) of 3 mM AMP-PNP.

FIG. 3. Effect of the ATP analog AMP-PNP on FP-GK binding activity. The binding activity was determined with 7.5 μM hGK, 20 mM glucose and 80 nM FL1. The non specific binding (NSB) was determined in the presence of 0.3 mM FL (added as 1 μl of 10 mM FL stock compound in DMSO to 30 μl reaction). 1 μl of DMSO was added to the other samples.

FIG. 4: Time course of the FP-GK binding reaction. An FP-GK binding assay was performed in 12 mM glucose, 1.5 mM AMP-PNP, 1.8 mM MgCl₂, 80 nM FL1, 0.23 μM hGK. Background samples contained 0.3 mM FL and Totals contained 1.6% DMSO.

FIG. 5: Effect of DMSO on the FP-GK binding activity.

FIG. 6: Assay window threshold with FL1 (40 and 80 nM) at different hGK concentrations. At each hGK concentration the first bar is NSB in the presence of 0.3 mM FL in DMSO and the second bar is in the presence of DMSO alone.

FIG. 7: Dose response of GK activator FL on FP-GK binding activity at different GK concentrations at 40 nM FL1 (A) or 80 nM FL1 (B) and 12 mM glucose.

### DEFINITIONS

The terms and phrases defined below are used throughout this application. Other terms not expressly defined would carry their normal and ordinary meaning as understood by those of ordinary skill in the art at the time of filing this application.

As used herein, “FP” corresponds to fluorescence polarization which relates to techniques based on the principle that a fluorescent labeled compound, when excited by linearly polarized light will emit fluorescence having a degree of polarization inversely related to its rate of rotation. The level of fluorescence can be measured and used to evaluate the activity in an assay.

As used herein “FL” corresponds to a glucokinase ligand (activates GK).

As used herein “FL1” corresponds to a fluorescently labeled FL ligand, preferably fluorescein labeled. FL is the parent compound of “FL1”

As used herein, “AMP-PNP” corresponds to Adenosine 5′-β,γ-imido) triphosphate, a non-hydrolyzable ATP analog).

As used herein “DTT” corresponds to dithiothreitol.

### EXAMPLES

The following examples describe specific embodiments of the subject matter described and claimed herein. These examples should be understood to be only specific embodiments and do not limit the scope of the invention(s) contemplated herein.

### Example 1

### Assay Validation

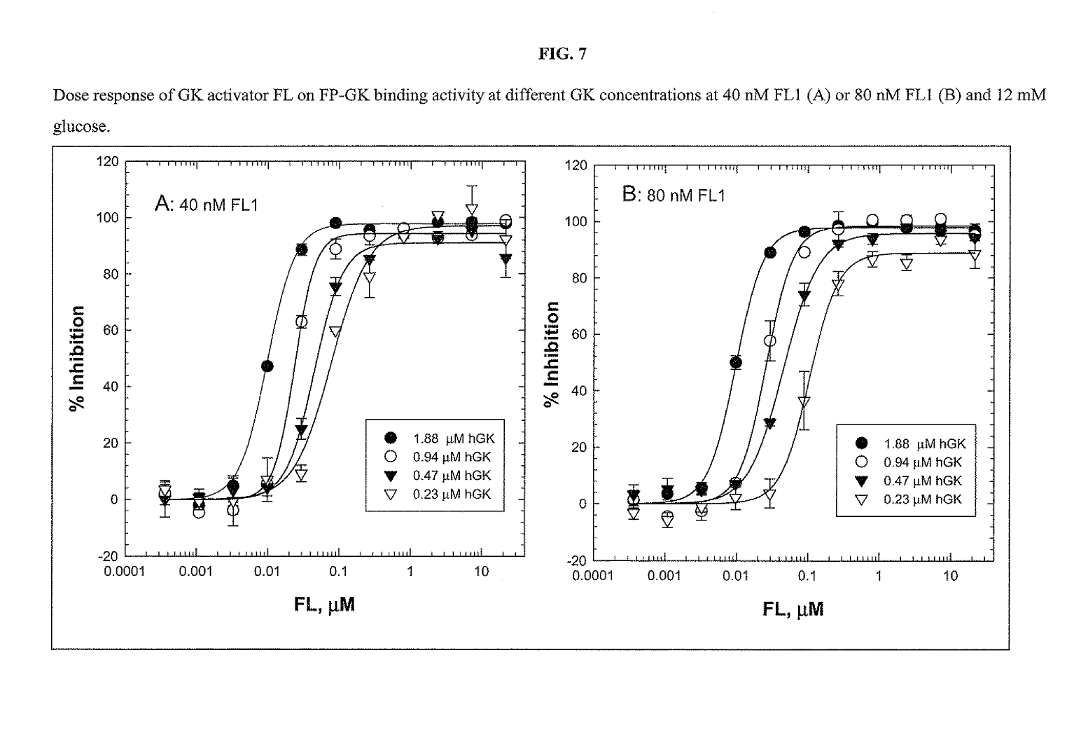

Full-length recombinant human GK was used to measure the binding affinity of FL and FL1 to GK. The optimal FP signal was obtained using freshly made buffer containing 25 mM HEPES, pH 7.1 (all subsequent solutions are made up in this buffer), 1 mM DTT, 3.6 mM MgCl₂. The wells of a CORNING® 384-well black plate (cat #3654) included hGK in 30 μL of assay buffer and the other assay reagents. FL and FL1 diluted in DMSO were added to all wells using a MULTIDROP® 384 Microplate Dispenser (Thermo-Labsystems, Helsinki, Finland). Following addition of the compounds, the assay plate was covered with aluminum foil seal and mixed on an orbital shaker. After incubation at room temperature for two hours, the seal was removed and the fluorescence associated with the wells/plates was measured using an ANALYST® plate reader. The data obtained from the plate reader was processed in XL-fit to calculate the IC₅₀, Yₘₐₓ obs, and slope values. XL-fit IC₅₀ curves are described in FIG. 7 and the IC₅© values from FIG. 7 are given in Table 2.

### Example 2

### Synthesis and Determination of GK Activation of Fluorescein Labeled Ligand

The fluorescein-labeled ligand FL1 was made by conjugating a fluorescein moiety to FL. FL and FL1 were tested for their activity in a GK activation assay. GK activity was assayed using a tandem GK-glucose 6 phosphate dehydrogenase (G6PDH) coupled discontinuous assay (described in U.S. application Ser. No. 11/869,778, which is incorporated herein by reference). FL and FL1 were (1 μl) dissolved and diluted in DMSO and added to the wells of black 384-well plate (CORNING® catalog #3655). 15 nM GK and 0, 2, 5, 12 or 20 mM glucose were then added to the plate in assay buffer (25 mM Hepes, pH 7.1, 3 mM ATP, 3.6 mM MgCl₂ and 1 mM DTT). The reaction was initiated by the addition of 4 μl of 15 mM ATP and incubated for 10 minutes. The reaction was quenched with 5 μl of 225 mM EDTA. The G6P formed as a result of the reaction was assayed by adding 25 μl of coupling reaction mixture containing 0.175 units G6PDH and 677 μM of thio-NAD. After a 15 minute incubation, the thio-NADH formed was detected by reading the absorbance at 405 nM in a SPECTRAMAX® Plus³⁸⁴ plate reader (Molecular Devices).

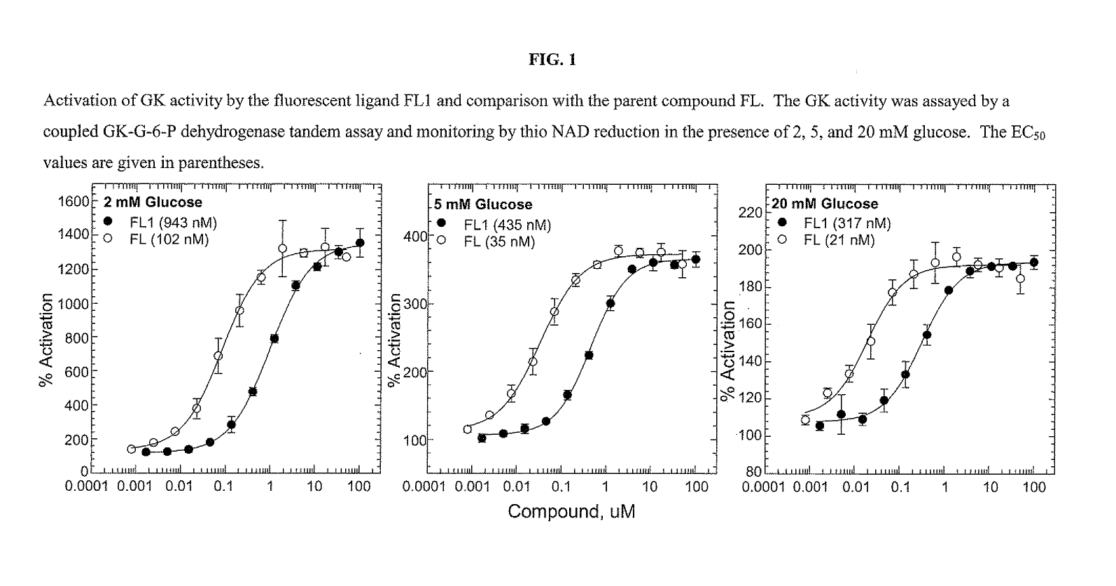

The data obtained from the GK activation assay reflect that FL and FL1 activated GK by about 200% with EC₅₀ values of 21 and 317 nM at 12 μM glucose, 35 and 455 nM at 5 mM glucose and 102 and 943 nM at 2 mM glucose respectively. (See FIG. 1). FL1 retained most of the GK activation activity of the parent compound (FL) but FL1, did, however, have reduced potency relative to the parent compound. One possible explanation for the reduced potency is due to the steric hindrance of the fluorescein moiety incorporated onto a thiazole group of the FL compound. These results show that FL1 has GK activator activity and may be effectively used for binding studies in looking for GK modulators (e.g., activators).

### Example 3

### Characterization of FL1 and Assay Parameters for an FP-GK Binding Assay

The following experiments were conducted to assess the feasibility of using a fluorescein labeled GK activator as a ligand in an FP-binding assay to characterize the activity of agents that bind to GK. As described above, the FL1 ligand has reduced affinity for GK and the experiments helped generate data to evaluate whether the FL1 ligand provides an effective range suitable for use in an FP binding assay.

To confirm that the interaction of GK and the fluorescent ligand would generate a response in the FP assay with an effective signal, 7.5 μM of GK was incubated in the binding buffer with FL1. Following incubation, the FP signal increased from 100 to 350 mP indicating that FL1 is an effective ligand for use in the binding assay with a dynamic range of 100 to 350 mP. signal of 100 mP in an FP assay is Typically, a Δ considered an effective assay. The FP assays signal of 250 mP which suggests the GK FP described herein using FL1 have a Δ binding assay is a robust and effective assay for measuring GK binding characteristics.

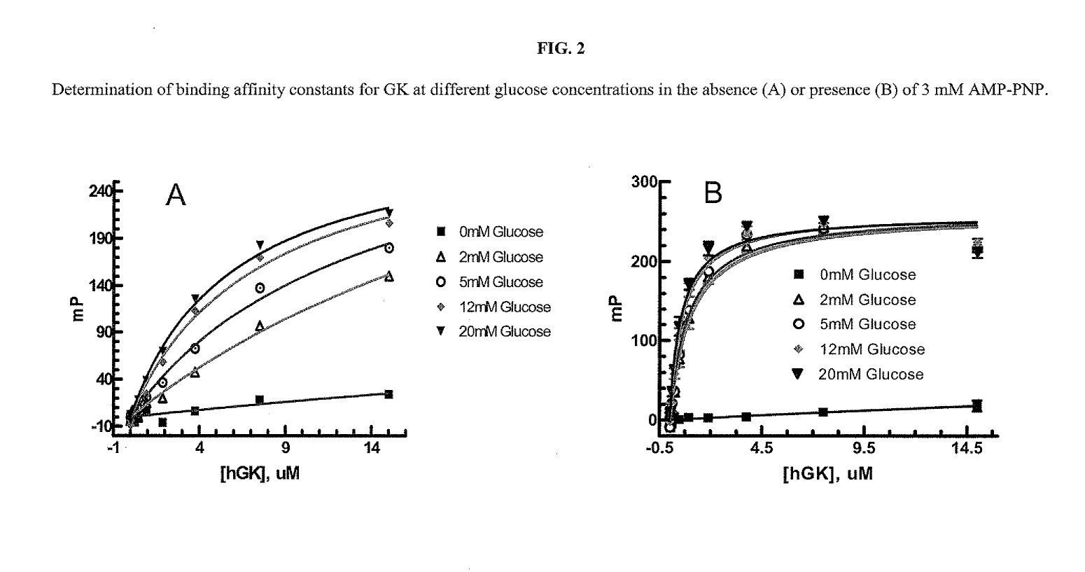

To determine whether FL1 binding to GK is glucose dependent, FL1-GK binding was titrated with GK (2× serial dilutions from 15 uM GK) in the presence of 0, 2, 5, 12, or 20 mM glucose in the presence or absence of 3 mM of an ATP analog AMP-PNP with 80 nM FL1. There was no significant FL1 binding in the absence of glucose or in the absence of AMP-PNP. The Kd for GK binding was 33.5, 13.41, 7.39, and 5.85 uM at 2, 5, 12 and 20 mM glucose, respectively, in the absence of an ATP analog (See data in FIG. 2A). There was no significant FL1 binding to GK in the absence of glucose even in the presence of 3 mM AMP-PNP. Similarly, the potency of FL1 binding to GK increased with increasing glucose concentration as evidenced by a reduced Kd with increasing glucose concentration in the reaction (See the data in FIG. 2B, Table 1). The absence of binding of FL1 to GK in the absence of glucose either in the absence or presence of AMP-PNP and decrease in binding Kd for protein with increasing glucose concentration suggests that substrate glucose is required for FL1 binding to GK.

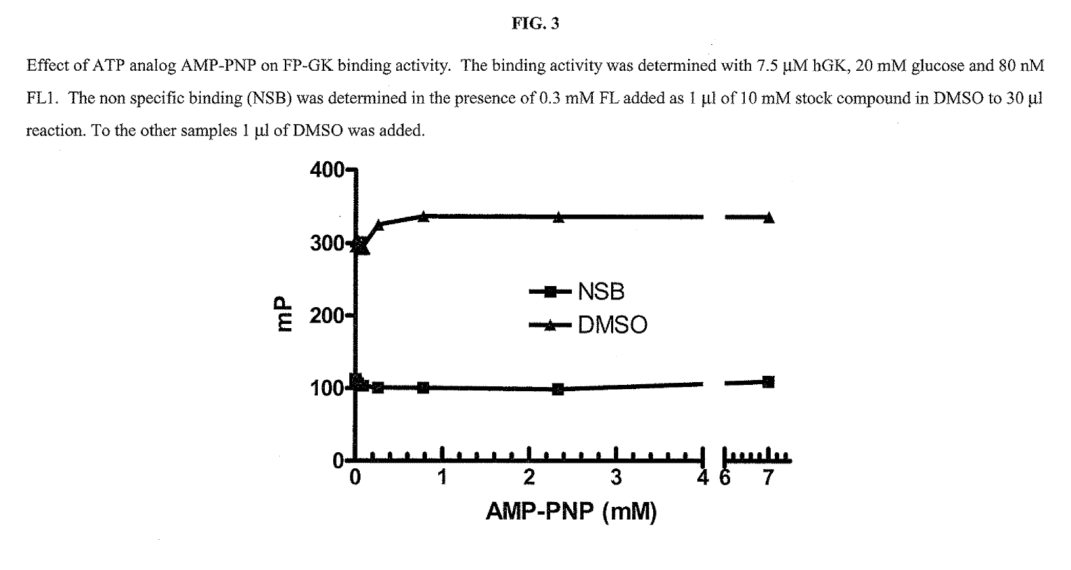

To determine the role of ATP in FL1-GK binding, a non-hydrolyzable ATP analog AMP-PNP (Sigma, St Louis, Mo.) was tested in the FP binding assay. Using the above described FP assay, the binding of FL1 to GK was measured and titrated with AMP-PNP at 12 mM glucose, 7.5 μM hGK and 80 nM FL1. AMP-PNP increased the binding by 30-40 mP in the presence of 0.26 mM AMP-PNP and remained steady up to 7 mM AMP-PNP (See data in FIG. 3). When binding of FL1 was titrated with hGK in the absence and presence of 3 μM. AMP-PNP, the Kd for GK decreased by more than 10-fold in the presence of AMP-PNP (See data in FIG. 2). The average Kd was 1.02, 0.90, 0.63, and 0.55 μM at 2, 5, 12 and 20 mM glucose respectively in the presence of AMP-PNP (Table 1). This suggests that AMP-PNP increased the affinity of ligand binding to the protein GK.

TABLE 1
Dependence of FP-GK binding affinity of ligand on AMP-PNP

| | 0 mM | 2 mM | 5 mM | 12 mM | 20 mM |
|---|---|---|---|---|---|
| Assay | Glucose | Glucose | Glucose | Glucose | Glucose |
| Condition | K_{d} (μM) | K_{d} (μM) | K_{d} (μM) | K_{d} (μM) | K_{d} (μM) |
| (−) AMP-PNP | >15 | 33.46 ± 0.13 | 13.41 ± 0.13 | 7.39 ± 0.07 | 5.86 ± 0.08 |
| (+) 3 mM | >15 | 1.02 ± 0.22 | 0.90 ± 0.09 | 0.63 ± 0.08 | 0.55 ± 0.09 |
| AMP_PNP | | | | | |

Note:
Average of 2-3 independent experiments performed in duplicate.

### Example 4

### Determination of Stability of FP Signal

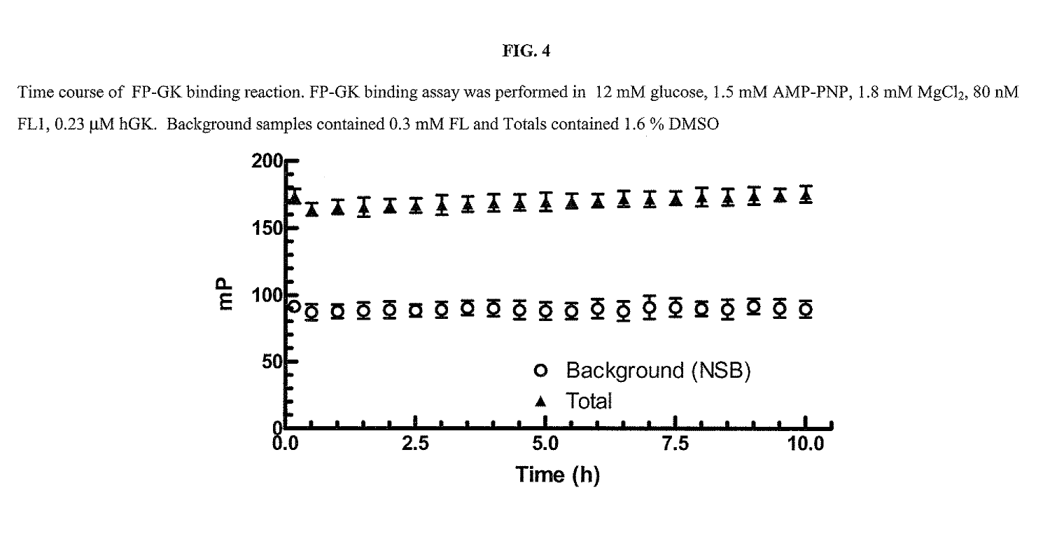

A time course analysis was conducted to determine the stability of the fluorescence signal in the FP-GK binding assay. The assay was conducted with 0.23 μM GK, 3 mM AMP-PNP, 12 mM glucose and 1 mM DTT and the blank was determined in the presence of 0.3 mM FL. The reaction was started with the addition of 80 nM FL1 and the FP signal was read at 10 minutes, 30 minutes, 1 hour and then every hour. The binding of FL1 to hGK was both rapid and relatively stable. As reflected in FIG. 4, within ten minutes, the FP signal reached maximum value, and remained constant thereafter for about ten hours. The stability of the FP signal over a long period of time provides an added advantage in high throughput applications.

### Example 5

### DMSO Tolerance in FP Binding Assay

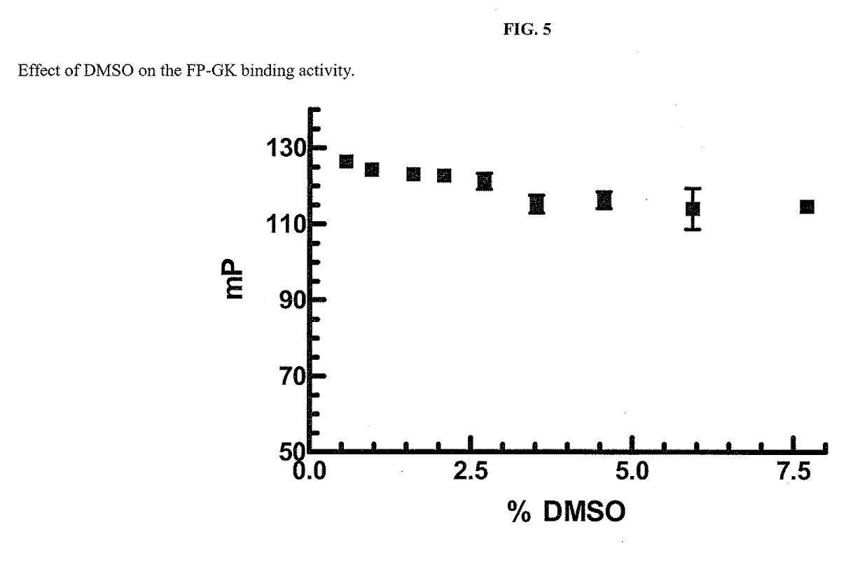

The effect of DMSO concentration on assay performance is an important factor in conducting high-throughput screens because compounds are routinely solubilized and diluted in DMSO. Therefore, the effect of DMSO on the FP-GK binding assay performance was evaluated by determining the FP signal at various DMSO concentrations. DMSO was diluted in assay buffer and 10 μl added to each well followed by 15 μl of enzyme mixture that gives final concentration of 0.47 μM GK, 3 mM AMP-PNP, 12 mM glucose, 3.6 mM MgCl₂ and 1 mM DTT. The reaction was started with the addition of 5 μl of 240 nM FL1 (final concentration 40 nM). After a 15 minute incubation, the FP signal was read in ANALYST®. As shown in FIG. 5, the FP-GK binding assay tolerated up to 2.7% (v/v) of DMSO, with less than 4% inhibition of binding activity. Notably, the binding activity was inhibited by about only 10% at 7.7% (v/v) of DMSO. The increased DMSO tolerance allows for a broader range of compounds to be tested because for lower affinity compounds a higher DMSO concentration can be used.

### Example 6

FP-GK Binding Assay Statistics

To determine the reproducibility and the robustness of the FP-GK binding assay, the statistical parameter Z′ was determined on three separate days using the equation set forth below (Zhang et al., “A simple statistical parameter for use in evaluation and validation of high-throughput screening assays”, J. Biomol. Screen, 4:67-73 (1999)).

$${Z}^{′} = 1 - \frac{( 3 {σ}_{bound} + 3 {σ}_{free} )}{( {mP}_{bound} - {mP}_{free} )}$$

where σ_{bound} is the standard deviation of FL1 conjugate; σ_{free} is the standard deviation of free FL1; mP_{bound} is the average FP value of FL1 conjugate; and mP_{free} is the average FP value of free FL1.

To determine the statistics for the assay, the assay was run in one 384 well plate containing totals (0.234 μM hGK, 80 nM FL1 in assay buffer) in 192 wells and nonspecific binding (10 μM FL, 0.234 μM hGK, 80 nM FL1 in assay buffer) in 192 wells in a checker board fashion on each day for three days. The average Z′ value for the FP-GK binding assay was 0.63 which suggests that this assay is a robust and high quality assay and therefore is suitable for automation and HTS.

### Example 7

### Evaluation of FP-GK Assay for Screening

To evaluate whether the FP-GK binding assay can be successfully used for identifying novel ligands, the parent compound FL, a known GK agonist, was used in the FP competition assay at 40 and 80 nM concentrations of FL1 and at different GK protein concentrations (0.23, 0.47, 0.94, 1.88 μM hGK; see data in Table 2).

TABLE 2
Dependence of IC₅₀ of FL on hGK Concentration
in FP-GK Binding Assay at 40 and 80 nM Ligand

IC₅₀, nM (n = 2)

| [hGK], μM | 40 nM FL1 | 80 nM FL1 |
|---|---|---|
| 1.88 | 916 ± 231 | 868 ± 117 |
| 0.94 | 374 ± 73 | 340 ± 59 |
| 0.47 | 173 ± 20 | 167 ± 10 |
| 0.23 | 100 ± 4 | 84 ± 25 |

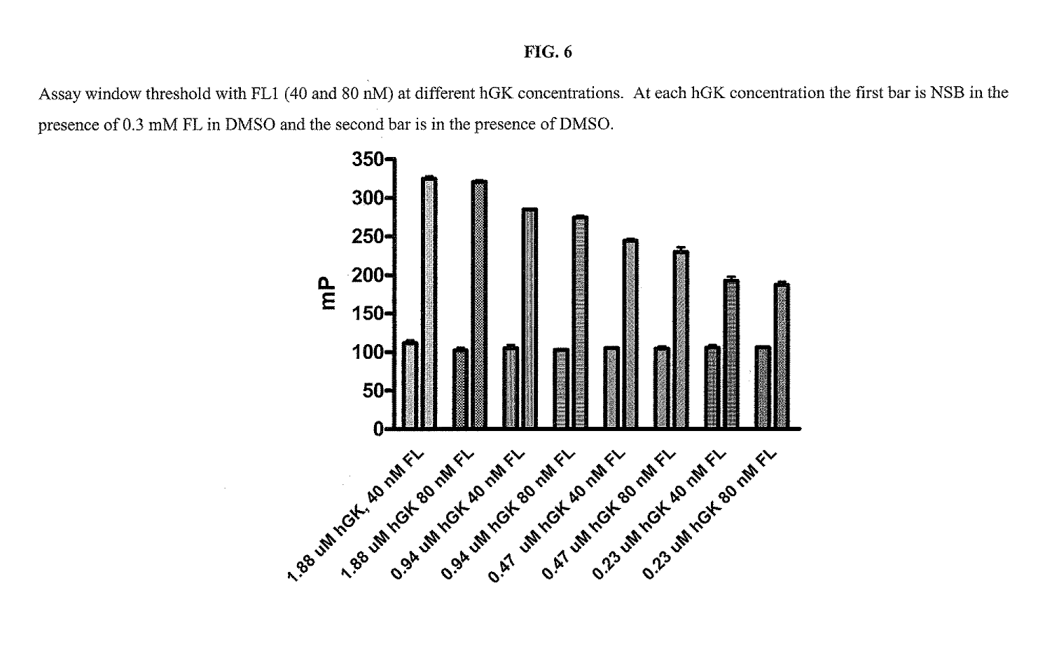

Varying the ligand concentration at the same hGK concentration did not significantly change the IC₅₀ value of FL. The signal decreased with decreasing hGK protein concentration whereas the background did not change with changing hGK concentrations (See data in FIG. 6). However, the IC₅₀ value for FL, increased with increasing concentrations of hGK protein at the same ligand concentration (See FIG. 7). These results suggest that, in the FP binding assay the IC₅₀ values of the compounds are independent of ligand concentration (when ligand was used below the Kd for the ligand), but depend on the enzyme concentration and showed a decrease with decreasing enzyme concentration. In all of the FP competition binding assays, 80 nM FL1, 3 mM AMP-PNP, 12 mM glucose and 0.23 μM of hGK protein were used unless described otherwise, because the IC₅₀ values obtained for FL and other activator compounds were lowest at 0.23 μM of hGK and correlated very well with EC₅₀ values obtained in GK activation.

### Example 8

### Validation of the Human FP-GK-Binding Assay

To evaluate whether the FP-GK binding assay can be successfully used for determining binding affinity of GK activators, we have evaluated a panel of GK activators with different EC₅₀ values in the FP-GK binding assays (assay conditions used: 0.23 μM hGK, 12 mM glucose, 3 mM AMP-PNP and 80 nM FL1). In the FP-binding assay, the relative affinity of a compound is determined by its ability to compete with FL1. The binding IC₅₀ values for these compounds were calculated using the 4-parameter logistic fit in XL-fit.

The binding IC₅₀ values obtained for these compounds by the FP-binding assay were compared against the EC₅₀ values obtained by the activity assay. As shown in Table 3, the FP-binding assay IC₅₀ values correlated very well with the binding K_{d} values obtained by the ITC method (Isothermal Titration Calorimetry—reference: Sigurskjold, B. W., “Exact analysis of competition ligand binding by displacement isothermal titration calorimetry”, Anal. Biochem., 277:260-266 (2000)). Also, binding IC₅₀ values obtained by the FP-GK binding assay correlated with the activation EC₅₀ values obtained by the GK activity assay. Compound instability or differences in the assays (these assays were not run concurrently) could have contributed to the minor discrepancies between the binding IC₅₀ values and activation EC₅₀ values for the compounds.

Overall, these results confirm that the FP-binding assay using a fluorescein-labeled FL1 ligand for hGK can be used successfully for determining the relative affinities of novel GK activators.

TABLE 3
Comparison of Activation EC₅₀ of GK Activators with FP-GK
Binding IC₅₀ and Binding Affinity Determined Using an ITC
(Isothermal Titration Calorimetry) Method

| | GK Activity | | FP-GK Binding |
|---|---|---|---|
| | Assay EC₅₀ | | Assay IC₅₀ |
| Test | 12 mM Glucose | ITC K_{D} | 12 mM Glucose |
| Compound | (μM) | (μM) | (μM) |
| A | 0.87 | | 1.23 ± 0.07 |
| B | 1.02 | 1.2 | 1.80 ± 0.47 |
| C | 1.33 | | 1.22 ± 0.15 |
| D | 0.19 | 0.24 | 0.32 ± 0.02 |
| E | 0.96 | | 0.55 ± 0.01 |
| F | 0.75 | | 0.54 ± 0.14 |
| G | 1.41 | | 2.09 ± 0.32 |
| H | 0.06 | | 0.13 ± 0.02 |
| I | 0.76 | | 0.37 ± 0.10 |
| J | 2.15 | 0.32 | 0.84 ± 0.13 |
| FL* | 0.04 | | 0.15 ± 0.05 |
| K | 0.26 | 0.39 | 0.52 ± 0.33 |
| L | 0.60 | | 0.82 ± 0.14 |
| M | 0.05 | 0.049 | 0.14 ± 0.01 |
| N | 0.03 | 0.039 | 0.07 ± 0.01 |

What is claimed is:

1. A method for measuring the binding characteristics of glucokinase (“GK”) comprising: incubating assay buffer, glucokinase, a fluorescently labeled GK ligand, in a test reaction vessel; incubating assay buffer glucokinase, and a control media in a control reaction vessel; measuring the fluorescence polarization (FP) signal in said reaction vessels; and comparing the FP signal in the reaction vessels whereby a difference in FP signal indicates binding to GK.

2. The method of claim 1 wherein said assay buffer comprises glucose and ATP analog, AMP-PNP.

3. The method of claim 1 wherein the GK is recombinant human GK.

4. The method of claim 1 wherein said test reaction vessel and said control reaction vessel are multi-well microtiter plates.

5. The method of claim 1 wherein said fluorescently labeled GK ligand is labeled using fluorescein.

6. The method of claim 1 wherein said GK ligand comprises a thiazole group.

7. The method of claim 1 wherein said assay buffer comprises a non-hydrolyzable nucleotide analog.

8. A method for identifying agents that bind (“GK”) comprising: a) incubating assay buffer, glucokinase, a fluorescently labeled GK ligand, and a test compound dissolved in medium in a test reaction vessel; b) incubating assay buffer, glucokinase, a fluorescently labeled GK ligand, and control media in a control reaction vessel; c) measuring the fluorescence in said reaction vessels; and d) comparing the fluorescence in the reaction vessels whereby a difference in fluorescence indicates binding to GK.

9. The method of claim 8 wherein said assay buffer comprises glucose and AMP-PNP.

10. The method of claim 8 further comprising a positive control wherein said positive control is known to bind GK and said comparing step d) includes a positive control in the comparison.

11. The method of claim 8 wherein said test compound has an IC₅₀ of from about 0.001 to about 2.5 μM in the presence of 12 mM glucose.

12. The method of claim 8 wherein the GK is recombinant GK.

13. The method of claim 8 wherein said GK concentration is varied.

14. The method of claim 8 wherein said test compound competes with said fluorescently labeled GK ligand.

15. The method of claim 8 wherein said fluorescently labeled GK ligand is labeled using a label selected from the group consisting of fluorescein, BODIPY, rhodamine green, rhodamine red, tetramethylrhodamine, alexa fluor, OREGON GREEN®, TEXAS RED®.
